(19) United States
(12) Patent Application Publication
(10) Pub. No.: US 2024/0141274 A1
(43) Pub. Date: May 2, 2024

(54) TILTING DEVICE FOR MICROSCOPY

(71) Applicant: ibidi GmbH, Gräfelfing (DE)

(72) Inventor: Valentin KAHL, Martinsried (DE)

(21) Appl. No.: 18/353,514

(22) Filed: Jul. 17, 2023

(30) Foreign Application Priority Data
Jul. 22, 2022 (EP) 22186407.7

(51) Int. Cl. C12M 3/06; C12M 1/34; C12M 3/00; G02B 21/26

(52) U.S. Cl. CPC C12M 27/16; C12M 23/48; C12M 41/00; C12M 41/44; G02B 21/26

(57) ABSTRACT

The invention relates to a tilting device for microscopy, comprising a sample carrier mount and an optical component with an optical axis, where the optical axis is aligned with respect to a base side of the sample carrier mount, where the sample carrier mount and the optical component are mounted to be tiltable, and where the alignment of the optical axis with respect to of the base side of the sample carrier mount is maintained when the sample carrier mount and the optical component are tilted.

The invention relates to a tilting device for microscopy and a microscope comprising the tilting device, as well as an associated method and use.

There are differently designed sample carriers for living cells in which the cells can be cultured and at the same time examined under the microscope. There are also sample carriers in which cells can be cultured under flow, or the medium in which the cells are cultured can be easily exchanged. In order to realize such a flow in a sample carrier, sample carriers typically have a channel system formed therein which comprises at least one inflow and one drain and is otherwise closed. Sample carriers with channel systems are known, for example, from EP 1 458 483 A2, EP 1 397 483 A1 and EP 4 030 216 A1. Such sample carriers are generally configured for microscopic examinations so that cells that are cultured in the channel system can be examined under a microscope.

In order to exchange the medium, in particular a fluid, in the channel system, a pump system or external reservoirs can be used, to which the sample carrier is connected, for example, by way of tubes. Such a pump system is disclosed, for example, in EP 1 944 084 A1. In the case of external reservoirs, fluid exchange can be achieved using exclusively passive components by way of different filling levels in the reservoirs and the principle of communicating tubes. However, both options require an additional device apart from the sample carrier itself in order to accomplish a flow within the channel system for exchanging the medium. Alternatively, the sample carrier itself can be tilted to create a flow within the channel system by gravity. A drawback of this solution becomes apparent with simultaneous tilting and microscopy. The tilting changes the distance between an objective of a microscope and the sample carrier, or between the objective and the region within the sample carrier to be examined under the microscope, respectively, so that the objective must be refocused accordingly.

In view of the drawback mentioned, it is an object of the present invention to provide a tilting mechanism for microscopy, a microscope, as well as an associated method and use that make it possible to improve microscopy for a sample carrier in which a flow is to be generated or to be present. This object is satisfied by the tilting mechanism according to claim 1, the microscope according to claim 10, the method according to claim 13, as well as the use according to claim 15. Further developments can be found in the respective dependent claims.

According to the invention, a tilting mechanism for microscopy is provided, comprising
a sample carrier mount and
an optical component with an optical axis,
where the optical axis is aligned with respect to a base side of the sample carrier mount,
where the sample carrier mount and the optical component are each mounted to be tiltable, and
where the alignment of the optical axis with respect to the base side of the sample carrier mount is maintained when the sample carrier mount and the optical component are tilted.

When the sample carrier mount and the optical component are tilted, the alignment of the optical axis of the optical component with respect to the sample carrier mount is maintained in the tilting mechanism according to the present invention during the tilting process. As a result, the optical component does not have to be adjusted to the tilting of the sample carrier mount, which enables improved microscopy at a sample carrier in a tilted sample carrier mount.

A tilting process is performed about a defined point or a defined axis. The tilting process can be executed clockwise or counterclockwise.

A sample carrier mount is a device configured to mount a sample carrier therein. The sample carrier mount has a base side that faces the optical component. The sample carrier mount can have in particular a base side with a recess through which microscopy can be performed and where no further element is arranged between the sample carrier and the optical component. Alternatively, the sample carrier mount can also be only a lateral frame in which a sample carrier is mounted, for example, by clamping, and which surrounds the sample carrier in part or entirely.

The invention also provides a tilting mechanism for a microscope, comprising
a sample carrier mount, and
an optical component with an optical axis,
where the optical axis is aligned with respect to a base side of the sample carrier mount,
where the sample carrier mount and the optical component are each mounted to be tiltable, and
where the alignment of the optical axis with respect to the base side of the sample carrier mount is maintained when the sample carrier mount and the optical component are tilted.

The base side of the sample carrier mount can be parallel to an underside of a sample carrier mounted in the sample carrier mount.

As a result, the alignment of the sample carrier is also parallel to the base side of the sample carrier mount, which serves as a reference value in the tilting mechanism. No optical correction that of a potential angular offset of the sample carrier, the actual object of microscopy, relative to the base side of the sample carrier mount then needs to be accounted for. An additional source of possible imaging errors is then eliminated.

The optical axis of the optical component is a line of symmetry through the optical component, for example, an axis of rotational symmetry of the optical component. In the case of mirrors or lenses, the optical axis passes through the respective center of curvature and is orthogonal relative to the remaining axes of symmetry of these optical components. At the same time the optical axis in geometrical optics defines an optical path on which light propagates through an optical system.

The optical component can be arranged below the sample carrier mount.

As a result, the tilting mechanism can be used in particular for use in the context of an inverted microscope.

The alignment between the optical axis and the base side of the sample carrier mount can be in particular vertical.

This special arrangement enables improved projection in microscopy using the tilting mechanism. In particular, coma, which is caused by light rays that are incident at an angle relative to the optical axis as a combination of astigmatism and spherical aberrations, is suppressed by the perpendicular alignment between the optical axis and the base side of the sample carrier mount.

The optical component can be an objective and/or a mirror.

Most microscopes, in particular transmitted light microscopes such as inverted microscopes, are constructed such that a collector lens, an objective, is directed at the object to be examined under the microscope. In the case of inverted microscopes, examinations are conducted at a sample carrier by directing the lens towards its base side. The tilting mechanism can therefore be integrated into an existing microscope and thus additionally provide the advantages described above.

Instead of a single mirror or lens, the optical component can also consist of several parts, for example, a doublet lens (such as an achromatic lens) or a triplet lens. A combination of mirrors and lenses is also possible.

The use of a multi-component optical component allows for the tilting mechanism to be more flexible, for example, in order to obtain more mechanical degrees of freedom or to carry out optical corrections as with an achromatic lens.

In particular, the optical component can comprise an objective as well as a mirror. In a special configuration, an objective is directed towards the base side of the sample carrier mount and is arranged between the sample carrier mount and a mirror. The mirror deflects light passing through the lens to a specific point of aim. When the sample carrier mount is tilted, the objective and the mirror tilt about a common axis of rotation so that the relative alignment of the mirror, of the objective, and of the sample carrier mount remains constant. The optical axis there corresponds to the optical path that, starting out from the sample carrier, runs through the lens and is deflected by the mirror by reflection. If the tilting mechanism is tilted about an axis that is perpendicular to the optical axis between the sample carrier mount and the objective as well as coaxial to the optical axis between the mirror and the point of aim, then the point of aim does not change due to the tilting process. In this case, the mirror deflects the light, in particular, at a right angle. Despite the tilting, the optical path between the mirror and the point of aim remains unchanged.

The arrangement of the mirror and the lens can also be in reverse order, in which case only the mirror can be considered as to be an optical component. In this configuration, the mirror is placed along the optical path between the sample carrier mount and the objective so that it redirects light coming from the sample carrier mount so that it is incident upon the objective. In this case, the lens is fixedly mounted and the mirror and the sample carrier mount are mounted to be tiltable and perform a tilt motion. If the tilting mechanism is tilted about an axis that is perpendicular to the optical axis between the sample carrier mount and the mirror, as well as coaxial to the optical axis between the mirror and the objective, and if the light is deflected by the mirror at a right angle, then a point of incidence and an angle of incidence of the light upon the objective due to the tilting do not change. Despite the tilting, the optical path between the mirror and the objective therefore remains unchanged.

An advantage of this second configuration is that the objective is fixedly mounted and does not move when the tilting mechanism is tilted. This enables a simpler design and in particular easier integration into an existing microscope that already comprises an objective. Furthermore, projection errors that arise as a result of a variation in the objective position over time can be reduced, which can result in improved projection quality.

The sample carrier mount and the optical component of the tilting mechanism can each be connected to a joint and can each be mounted to be tiltable about the respective joint.

This means that the optical component and the sample carrier are individually mounted to be tiltable, but that said alignment is retained during a tilting process. For this purpose, both components can be tilted independently of one another and mounted about their own tilt axis. The tilt axis of the optical component and the tilt axis of the sample carrier mount are then coaxial. This enables greater flexibility and numerous implementation options and possibilities for integrating such a tilting mechanism into a microscope.

A joint represents a mechanically established and reliable option for realizing tilts and rotations of certain elements.

The sample carrier mount and the optical component can be connected to the same joint and can be mounted to be tiltable about this joint.

Such a configuration represents a simple and reliable arrangement for realizing a tilting process of the optical component and the sample carrier mount in which the perpendicular alignment described between the optical axis and the base side of the sample carrier mount is maintained in the event of a tilting. In particular, this prevents an unwanted relative motion between the optical component and the sample carrier mount when tilting since both components are tilted together.

Typical examples of a joint there comprise ball joints and pivot joints or hinge joints. While rotations in a pivot joint can only take place about one axis, a ball joint allows for rotation about a plurality of axes that are arranged in one plane. A ball joint therefore provides greater flexibility with regard to the tilt axis. At the same time, a pivot joint allows for rotation about a well-defined axis and therefore provides better precision and stability. A pivot joint is suitable for the special configurations described above for the sample carrier mount and the optical component since it allows for rotation only about a well-defined axis.

The sample carrier mount and the optical component can be rigidly connected to one another.

A rigid connection between the sample carrier mount and the optical component is a simple way of maintaining the vertical alignment of the optical axis of the optical component with respect to the base side of the sample carrier mount when the two components mentioned are tilted. In addition, a rigid connection is characterized by a high degree of robustness so that the relative alignment of said components remains particularly stable. This is advantageous in the context of the precision required in microscopy because a relative motion between the optical component and the sample carrier mount cannot occur, which could lead to projection errors that may vary over time.

The tilting mechanism can be configured such that the sample carrier mount and the optical component are tilted about a tilt axis which is aligned to be perpendicular to the optical axis.

In other words, the tilt axis can be disposed in a plane that is perpendicular to the optical axis and the tilting process is conducted about this axis.

The constraint that the tilt axis is aligned to be perpendicular to the optical axis nevertheless allows for a high degree of flexibility in terms of tilting. For example, one can define a coordinate system around the tilting mechanism in which the optical axis is aligned along the Z-axis. In this case, the tilting process takes place about a tilt axis perpendicular to the Z-axis, i.e. for example, about the X-axis, the Y-axis, or any desired superposition of these two axes. In the latter case, the tilting mechanism has the highest possible number of degrees of freedom for tilting since rotation about the Z-axis corresponds to no tilting.

The tilting mechanism can be configured such that the sample carrier mount and the optical component of the tilting mechanism can be tilted by a tilt angle between 0 degrees and 1 degree, in particular between 0 degrees and 20 degrees, with respect to a horizontal position of the sample carrier mount.

In particular, the tilting mechanism can be configured such that the sample carrier mount and the optical component of the tilting mechanism can perform an oscillating circular motion. This is understood to mean a time-dependent tilt motion in which a vector parallel to the optical axis describes a circular elliptical trajectory. This motion is generated by a time-dependent superposition of a tilt about the X-axis and a tilt about the Y-axis.

The tilting mechanism allows for different applications depending on the range of tilt angles that can be achieved. Precise tilting can be performed for a small range between 0 degrees and 1 degree for examinations where only small but precise tilt angles are required. As described above, the tilting mechanism can be used to transport fluids in a sample carrier. In this manner, the flow rate can be adjusted particularly low and precisely so that cells can be examined under well-defined conditions. On the other hand, a larger range of tilt angles, for example, between 0 degrees and 20 degrees, increases the versatility of possible examinations that can be conducted. At the same time, a fluid, for example, a cell medium, can be exchanged quickly. A circular flow can be created in a sample carrier with an oscillating circular motion.

The tilting mechanism can furthermore comprise a motion device, where the motion device is configured such that it can move the optical component in a plane parallel to the base side of the sample carrier mount, or where the motion device is configured such that it can move the sample carrier mount in a plane perpendicular to the optical axis.

In this case, the motion device can move the sample carrier mount whereas the optical component is fixedly mounted. Alternatively, the motion device can move the optical component whereas the sample carrier mount is fixedly mounted.

The motion device can also be configured such that it can move the optical component perpendicular to the base side of the sample carrier mount or parallel to the optical axis of the optical component, respectively, or that it can move the sample carrier mount parallel to the optical axis of the optical component.

In this case as well, the motion device can move the sample carrier mount whereas the optical component is fixedly mounted. Alternatively, the motion device can move the optical component whereas the sample carrier mount is fixedly mounted.

This embodiment does not apply to a tilting mechanism in which the optical component and the sample carrier mount are rigidly connected to each other.

A relative motion between the optical component and the sample carrier mount in a plane parallel to the base side of the sample carrier mount ensures that a distance between the optical component and the sample carrier mount or the vertical alignment between the optical axis and the base side of the sample carrier mount does not change. At the same time, it is possible to examine at different points a sample carrier introduced into the sample carrier mount, in particular also while a tilt motion is being performed. In this case, it is not necessary to adjust the optical component, in particular to refocus an objective.

Furthermore, a relative motion between the sample carrier mount and the optical component parallel to the optical axis additionally allows for a focus of the optical component to be at different depths within a sample carrier. In addition to the horizontal relative motion between the sample carrier mount and the optical component described above, a complete three-dimensional examination of a sample carrier is therefore now possible. The advantages of the tilting mechanism described above apply in the same manner.

In this case, the motion device can be controllable, for example, manually. For this purpose, for example, the sample carrier mount or the optical component can be connected to a translation stage. A motion of the connected component can be obtained manually with a corresponding set screw of the translation stage. The motion device can also comprise a locking screw or a comparable element with which the motion device can be locked off after the adjustment has been made so that a further relative motion between the optical component and the sample carrier mount is ruled out.

Conventional translation stages typically allow for a precision of the adjustment in the range of a few tens of micrometers, which is sufficient for most applications in microscopy.

At the same time, the motion device can also be motorized, for example, in the form of a motorized translation stage. Other than that, the same considerations apply as for a manual embodiment. It is understood that an associated control device is required in the case of a motorized motion device. This shall be described hereafter. A motorized motion device entails several advantages. Firstly, the resolution and precision of the adjustment are typically higher than with a manual configuration. In addition, the motorized motion device can be automated and controlled externally, for example, by way of an external control device such as a computer. In addition to precise control of the relative position between the optical component and the sample carrier mount, this also enables motion control executed in a time-controlled and automated manner.

The tilting mechanism can further comprise an inclination device with a motor,
where the motor is connected to the optical component and/or the sample carrier mount, and
where the motor is configured such that it tilts the sample carrier mount and the optical component.

This inclination device allows for the tilt angle to be adjusted in an automated manner, in particular by way of an external control device such as a computer. In general, this allows for greater precision than manual adjustment. Furthermore, the microscopic observation can be carried out simultaneously with the tilting and does not have to be interrupted for this.

In particular, the configuration of the inclination device, in particular the shape of the motor, can depend on which tilt angle is to be obtained. A motor in the form of a piezoelectric actuator is ideal for very small tilt angles of around 1 degree or less. These enable the angle to be adjusted with very high precision and reproducibility. The electrical voltage required for the piezoelectric actuator can be provided by the external control device described.

However, a piezo-driven actuator is rather unsuitable for larger angles because the stroke required for this cannot be obtained. For example, an appropriately configured electric motor or a rotary motor is suitable instead for that purpose.

The inclination device can also be configured such that it generates a continuous tilt motion, in particular a seesaw motion of the tilting mechanism. A seesaw motion is a continuous tilt motion in which a direction of the tilt motion changes at well-defined time intervals. In order to achieve this, the inclination device must allow a time-dependent angular velocity in which in particular a change in the sign of the angular velocity is also possible.

Certain types of cells need to be cultured under a continuous flow. In this case, a tilt motion of the tilting mechanism and therefore also of a sample carrier that is mounted represents a possibility of generating this continuous flow without additional components. By changing the direction of the tilt motion, a fluid in the sample carrier can be kept in constant motion without additional fluid having to be refilled.

In addition to the motor-driven inclination device, it is also possible to allow for additional manual adjustment. For this purpose, the tilting mechanism can comprise, for example, a locking screw which can be loosened and tightened again by hand. This has the advantage, for example, that a large adjustment of the tilt angle can be made by hand, while the more precise adjustment is made by motor. As a result, the motor only has to be able to provide a smaller range of tilt angles.

The tilting mechanism can furthermore comprise a inclination sensor and a control device, where the inclination sensor is configured such that it can detect and output a value of a tilt angle of the tilting mechanism, and where the control device is configured to receive the value output by the inclination sensor and, based on the value received and a predetermined target value, to transmit a control signal to the inclination device so that the inclination device adjusts the tilt angle to the predetermined value.

On the one hand, this embodiment with an inclination sensor and a control device allows for the tilt angle to be adjusted in an automated manner, where the same advantages apply in this respect as in the case of automation of the motion device. In addition, these additional features also allow for a feedback mechanism that allows the tilt angle to be able to be stabilized at a specific value. As a result, examinations can also be conducted over long periods of time and under particularly stable and/or well-defined conditions.

Different embodiments are conceivable for the realization of an inclination sensor. In a simple form, an angle scale or a comparable visual marking can be visibly attached to the tilting mechanism, from which the tilt angle can be read. In addition, an inclination sensor can be attached to the tilting mechanism and can detect and electronically output the value of the tilt angle via, for example, three mutually perpendicular acceleration sensors or by way of a capacitive fluid sensor or a magnetoresistive sensor. This angle that is output can then be displayed by the external control device or output and/or used in some other way. In particular, the value of the tilt angle can be transmitted to the control device. This transmission can be conducted, for example, via radio, Bluetooth, wireless LAN, RFID, LAN or other known transmission mechanisms.

The control device can receive the value for the tilt angle that is output by the inclination sensor. The control device can there comprise a processor for determining or calculating the control signal on the basis of the value received and a predefined target value for the tilt angle. This control signal can subsequently be transmitted from the control device to the inclination device, where the same options are available for this transmission as between the inclination sensor and the control device.

The inclination device can receive the control signal and, based thereupon, control the motor such that the tilt angle is adjusted to the predetermined target value.

The control device can also be the external control device described, which also comprises the control device in addition to the inclination device. The external control device can comprise one or more processors for determining the control signals for the associated devices.

The tilting mechanism can also comprise compensation optics, for example, a movable mirror.

The compensation optics can be configured such that it compensates for the motion resulting from the tilting of the tilting mechanism such that the point of incidence and/or angle of incidence of light that passes through the sample carrier mount and the optical component remain unchanged even with a tilting process.

The moveable mirror can be a mirror mounted on a conventional motorized mirror mount and be moveable by way of a respective electrical signal. The electrical signal can there be provided, for example, by the external control device.

The additional compensation optics simplifies, among other things, the integration of the tilting mechanism into an existing conventional microscope. In addition to an objective, a microscope typically comprises at least one or more deflection mirrors and an eyepiece with which a virtual image is projected onto a sensor or the human eye. The tilting mechanism described can now be integrated, for example, into such a microscope in that the objective and a specimen stage are exchanged for the tilting mechanism described. Consequently, the tilting mechanism can be a replacement module with which a conventional microscope can be retrofitted. As a result of a tilting process, the optical path of light in the microscope changes. In order not to influence the imaging in the general case, the point of incidence and the angle of incidence of light that passes through the objective must remain unchanged as a result of the tilting. This can be achieved by the compensation optics described.

Furthermore, the present invention includes a microscope comprising a tilting mechanism as described above, a stand, and a projection system with a projection lens, in particular, an eyepiece.

The use of a described tilting mechanism in a microscope also leads to the advantages mentioned in the context of the tilting mechanism.

Inverted microscopes are typically preferably used in the context of the culturing and observation of cells in sample carriers provided for this purpose. The reason for this is that the cells are usually arranged on a bottom of the sample carrier. In an inverted microscope, the lens is directed at the object to be examined from below, so that the object can be approached particularly close to the cells. This enables higher optical resolution. However, the tilting mechanism presently described can also be employed in a microscope in which the objective is directed at the sample carrier from above, because the basic structure of the two types of microscopes mentioned only differs in the arrangement of the imaging components, but not in the components themselves.

In particular, the projection system comprises an eyepiece that produces a viewable image. In addition, further optical elements can be comprised in the projection system, for example, one or more mirrors or a prism for deflecting the light. The eyepiece itself can be made up of one or more lenses. It is possible to view the image formed through the eyepiece with the human eye.

The invention also provides a microscope comprising a sample carrier mount, and an optical component with an optical axis, a stand and a projection system with a projection lens, in particular an eyepiece, where the optical axis is aligned with respect to a base side of the sample carrier mount, where the sample carrier mount and the optical component are mounted to be tiltable, where the alignment of the optical axis with respect to the base side of the sample carrier mount is maintained when the sample carrier mount and the optical component are tilted, and where the projection lens is arranged between the optical component and the imaging component.

The projection system of the microscope can also comprise one or more mirrors, in particular movable mirrors, where the mirrors are coupled mechanically or can be controlled electronically individually. The projection system can comprise an imaging component, in particular a camera with an imaging sensor.

An imaging component in the form of a camera allows for automated and possibly continuous recording of image data with the microscope. This image data can be evaluated directly, for example, by a processor configured for this purpose.

The mirrors are used mainly to transmit an image from the objective to the imaging component. The mirrors can therefore be provided to deflect the light from the objective onto the imaging component, i.e. they are so-called deflection mirrors. The mechanical coupling of the mirrors or the electronic controllability there fulfill the purpose of adjusting the optical path of the light, which changes as a result of the tilting mechanism tilting, such that a point of incidence and an angle of incidence upon the imaging component remain unchanged.

The mechanical coupling can be formed between two or more mirrors so that these coupled mirrors perform a correlated motion. For example, the two or more mirrors can be connected directly to one another by a mechanical element.

At least one of the mirrors can also be mounted on a motorized mirror mount that can adjust the orientation of the mirror. In particular, the motor can be connected and controlled by a device provided for this purpose. This can be, for example, the external control device described above which can also control the inclination device and the control device. Individual control of the mirrors of the microscope allows for optimal control of the optical path through the microscope and therefore for precise adjustment of the orientation of the mirrors so that the projection is not affected by tilting.

Furthermore, the microscope can comprise a display device, in particular a monitor. This allows the image generated by the imaging component to be displayed.

The projection lens can be mounted to be movable along its optical axis, or can have a variable focal length, where a motion of the projection lens along its optical axis and/or the focal length is/are controllable individually.

Depending on the position of the axis of rotation when the tilting mechanism is tilted, its length can also change with the course of the optical path, which cannot be able to be compensated for by the mirrors described above. The consequence of this is that the imaging component is no longer in the focal plane of the projection lens and a blurred image is produced. By changing the focal length or the position of the projection lens, its focal plane (image plane) can be adjusted such that it always lies on the imaging component and a sharp image is produced.

The projection lens can be mounted to be movable, in particular in a rail, so that a motion only along the optical axis of the projection lens is possible. Together with the compensation optics and/or the mirrors of the microscope, there are a sufficient number of degrees of freedom present to compensate for a change in the optical path as a result of the tilting process, so that the projection is not changed. A motion of the projection lens perpendicular to its optical axis is not absolutely necessary for this.

The motion can be performed either manually or controlled by the external controller. The same considerations apply there as in the described setting of a tilt angle of the tilting mechanism using the inclination device.

In addition to or in place of the moveable mount, the projection lens can also have a variable focal length, such as a varifocal lens. In this case, a varifocal lens comprises a plurality of individual lenses which can be moved relative to one another, so that the overall focal length of the system changes. This lens therefore also solves the technical problem of compensating for a changed focus as a result of the tilt of the tilting mechanism. Here as well, the focal length can also be adjusted manually or by the external control device.

The variable position and the variable focal length of the projection lens can also be combined or employed together, respectively. In addition, the projection lens, in particular an eyepiece, can be configured having multiple lenses. In this case, one, several or all of the lenses can be arranged to be movable and/or have a variable focal length. Each of the positions and focal lengths of the lenses can be controlled and adjusted manually and/or by the external control device.

The mechanisms described above for adjusting the adjustment lens allow for great flexibility of the microscope. In a particular embodiment, it is possible for the optical component to be an objective corrected to infinity. The working distance of the objective is roughly the same as its focal length so that the light rays run substantially parallel downstream of the objective. Therefore, a deviation in the path length between the objective and the imaging component as a result of a tilting process does not necessarily lead to a different focus of the projection lens. A sharp image therefore continues to be generated on the imaging component and an adjustment of the projection lens is not necessary.

The microscope can be enclosed by an incubation system that regulates a temperature, gas concentration, and/or humidity at least at one location of the optical component and of the sample carrier mount. Such an incubation system is known, for example, from EP 2 148 921 A2.

An incubation system can set and control the culturing conditions for cells very precisely. This means that the observation as well as the culturing of the cells can be conducted in a common device.

The incubation system can be configured such that it forms a space, in particular an enclosed space, around the microscope. No structural changes need to be made to the microscope itself for integrating it into the incubation device.

The present invention further comprises a method. The method comprises the following steps of:
providing a tilting mechanism for microscopy as described above or a microscope as described above;
mounting a sample carrier in the sample carrier mount; and
tilting the sample carrier mount,
where the sample carrier comprises a first reservoir, a second reservoir, and a channel,
where the first reservoir is connected to the second reservoir by way of the channel,
where a fluid is transported from the first reservoir into the second reservoir by tilting the sample carrier mount and where, in particular, a flow is produced by the transport of the fluid, and where cells are cultured under this flow.

Complete performance of this method enables transporting a fluid within a sample carrier without the presence of an external device, for example, a pump device, but only as a result of the sample carrier being tilted. This transport of fluid creates a flow under which cells are cultured in this sample carrier. Such a flow is necessary, for example, to supply the cells with nutrients or to exchange used fluid. In addition, the method also provides the possibility of continuously observing the sample carrier while tilting is being carried out because the working distance and the alignment between the sample carrier and the optical component do not change and the optical component therefore does not need to be adjusted to the changed focus during microscopy. This allows for efficient microscopy.

A sample carrier is presently a stage for examining cells. In particular, the sample carrier can have a flat underside, which is particularly advantageous for microscopic examinations through this underside, especially in the case of inverse microscopy. Furthermore, the sample carrier is preferably made of transparent material, for example, plastic materials such as COC (cyclo-olefin copolymer), COP (cyclo-olefin polymer), PC (polycarbonate), PS (polystyrene), PE (polyethylene), PMMA (polymethyl methacrylate) or a transparent thermoplastic material or an elastomer or a mixture thereof.

Due to the use of the materials and methods mentioned, the sample carriers can be produced inexpensively and in large numbers having uniform quality. The reason for this is that injection molding with plastic materials is an established and reliable process and is particularly applicable in the case of the plastic materials mentioned. The use of transparent plastic material is particularly advantageous for being able to conduct optical examinations in the sample carrier, for example, by way of microscopy.

The method described can also be carried out with a tilting mechanism comprising an inclination device, where the sample carrier furthermore comprises a fluid sensor, and where the fluid sensor detects and outputs a value of one or more of a fluid level, a flow rate, or a flow direction within the sample carrier mounted in the sample carrier mount, and where the control device receives the value that is output by the sensor and, based on the value received, transmits a control signal to the inclination device so that the inclination device adjusts a tilt angle of the tilting mechanism so that one or more of the fluid level, the flow rate, or the flow direction within the sample carrier are adjusted to a predefined target value.

The culturing of cells in a sample carrier should preferably be conducted under well-defined environmental conditions, which in particular also comprise the stated parameters of fluid level, flow rate, and flow direction. These parameters can be controlled in a precise and stable manner and set by the control device in communication with the fluid sensor so that optimal and stable conditions for the culturing of cells can be provided in the sample carrier. In particular, the parameters mentioned can there be controlled in an automated manner so that the entire culturing process can also run in an automated manner without continuous control by a human operator.

In addition, the present invention comprises a use of a tilting mechanism described above for microscopy or a microscope described above for transporting a fluid within a sample carrier,
where the sample carrier is mounted in the sample carrier mount,
where the sample carrier comprises a first reservoir, a second reservoir, and a channel,
where the first reservoir is connected to the second reservoir by way of the channel,
where a fluid is transported from the first reservoir into the second reservoir by tilting the sample carrier mount and where, in particular, a flow is produced by the transport of the fluid, and where cells are cultured under this flow.

This is accompanied by the same advantages as in the method described above. This pertains in particular to the culturing of cells under a flow in a sample carrier and the continuous microscopy made possible when the tilting mechanism is tilted, because the focus of the optical component does not have to be corrected.

Further features and advantages shall be explained below using the exemplary figures, where

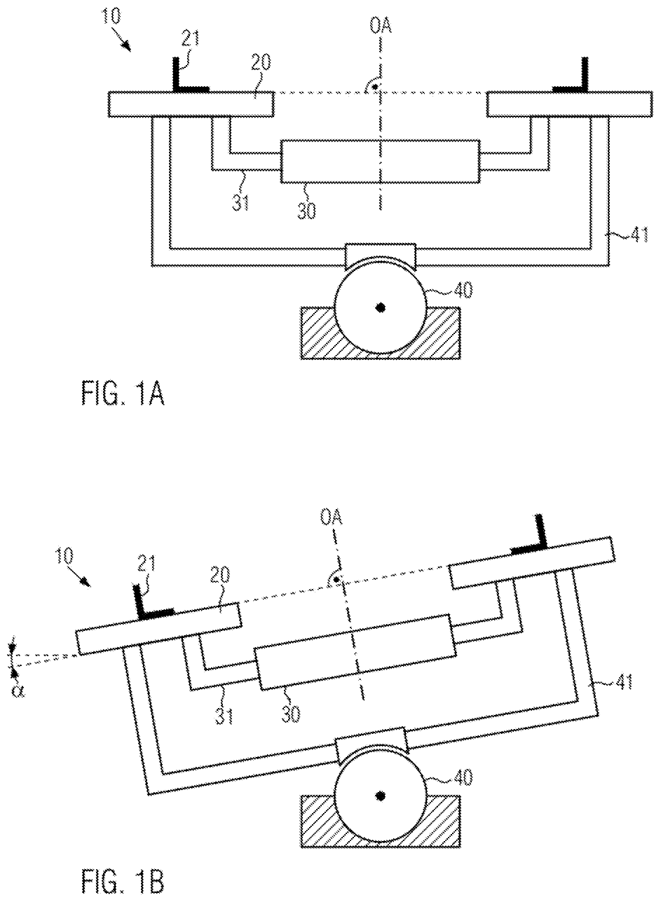

FIGS. 1A and 1B show a schematic cross section in a front view of a tilting mechanism according to a first and a second embodiment;

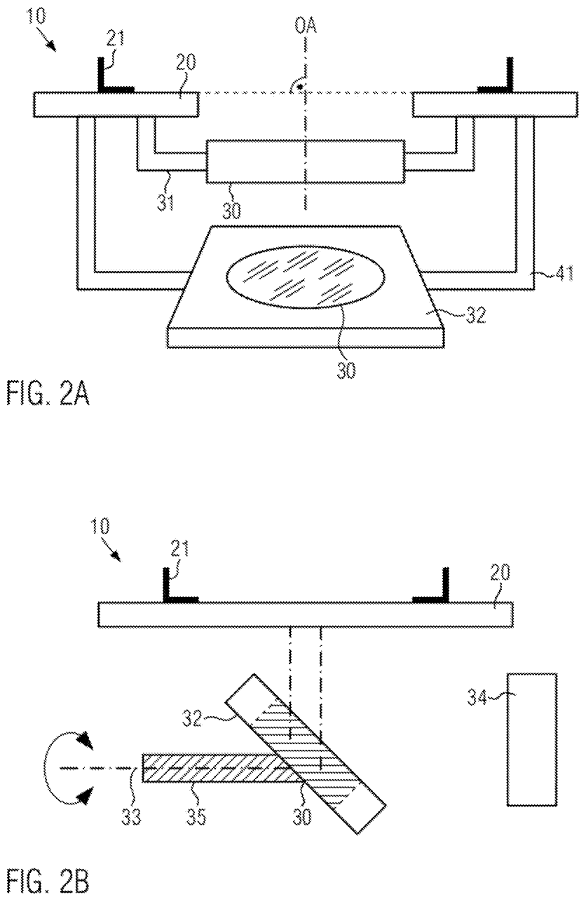

FIG. 2A shows a schematic cross section in a front view of a tilting mechanism according to a further embodiment;

FIG. 2B shows a schematic cross section in a side view of a tilting mechanism according to a further embodiment;

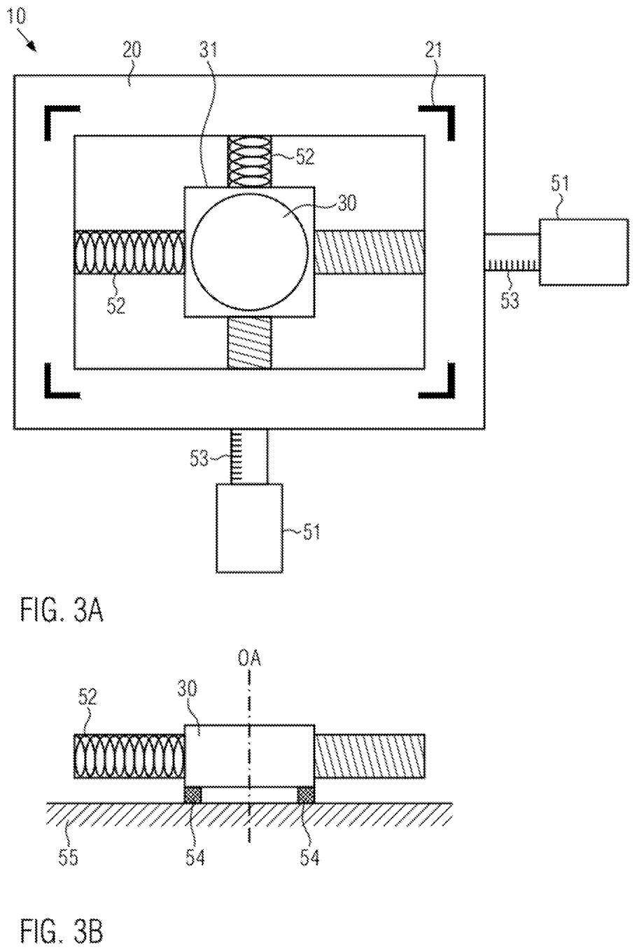

FIG. 3A shows a schematic tilting mechanism comprising a motion device as a top view;

FIG. 3B shows a schematic tilting mechanism comprising a motion device in cross-sectional view;

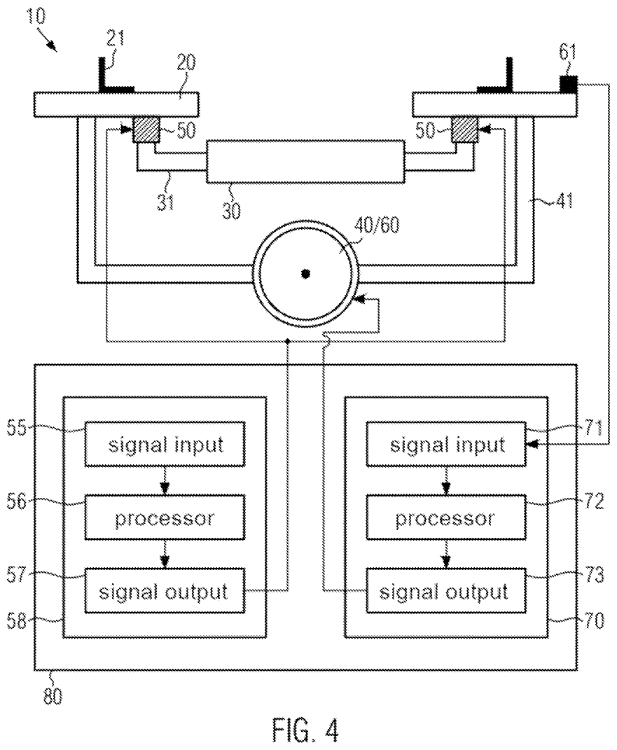

FIG. 4 shows a schematic cross section in a side view of a tilting mechanism according to a further embodiment;

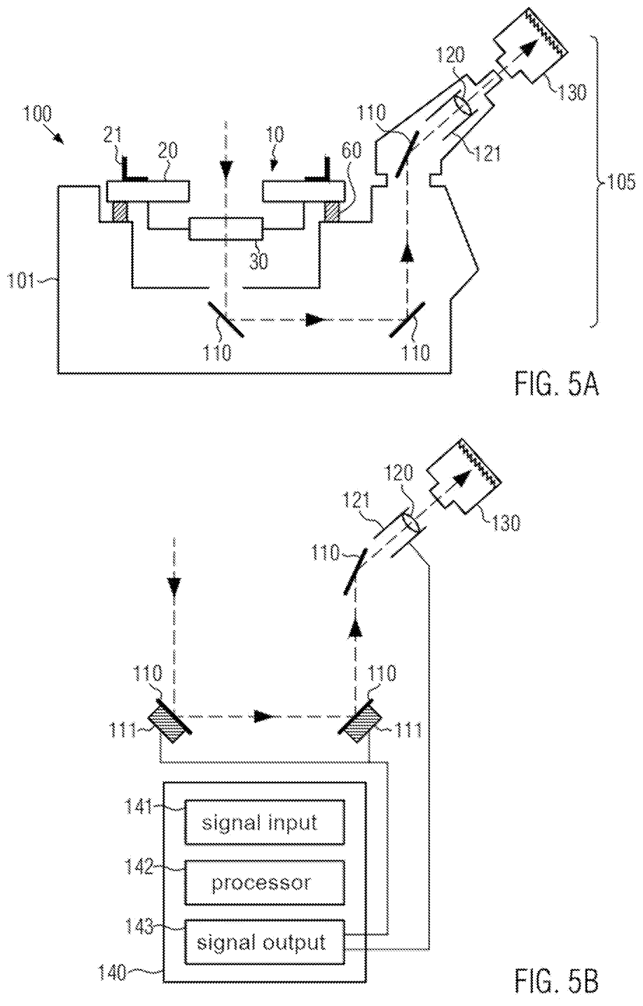

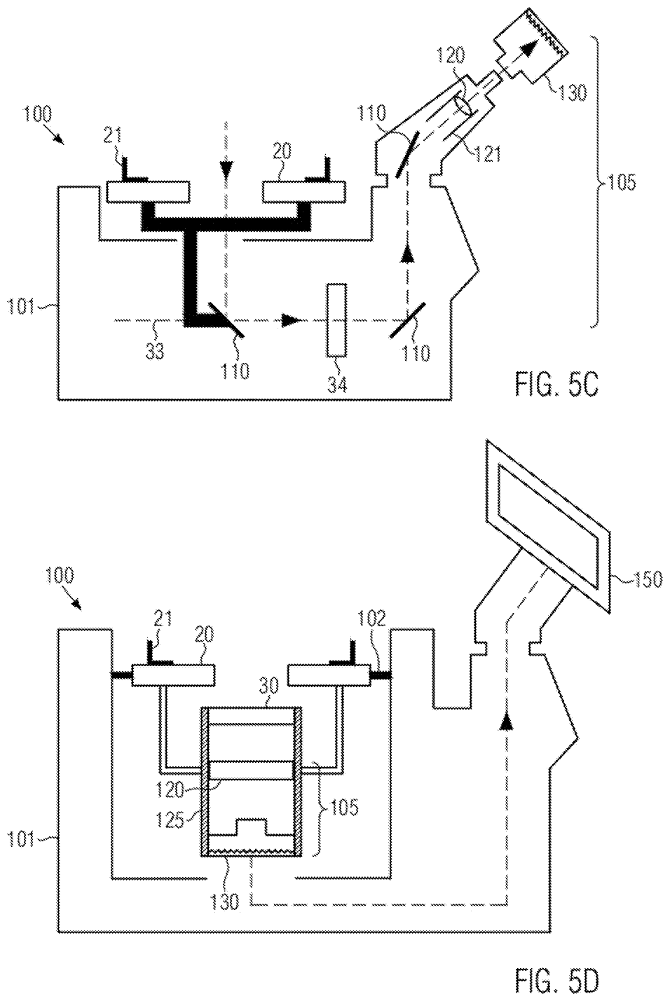

FIG. 5A shows a schematic microscope according to a first embodiment in cross section;

FIG. 5B shows a detailed view of the microscope in FIG. 5A;

FIG. 5C shows a schematic cross section of a microscope according to a further embodiment;

FIG. 5D shows a schematic microscope according to a further embodiment in cross section;

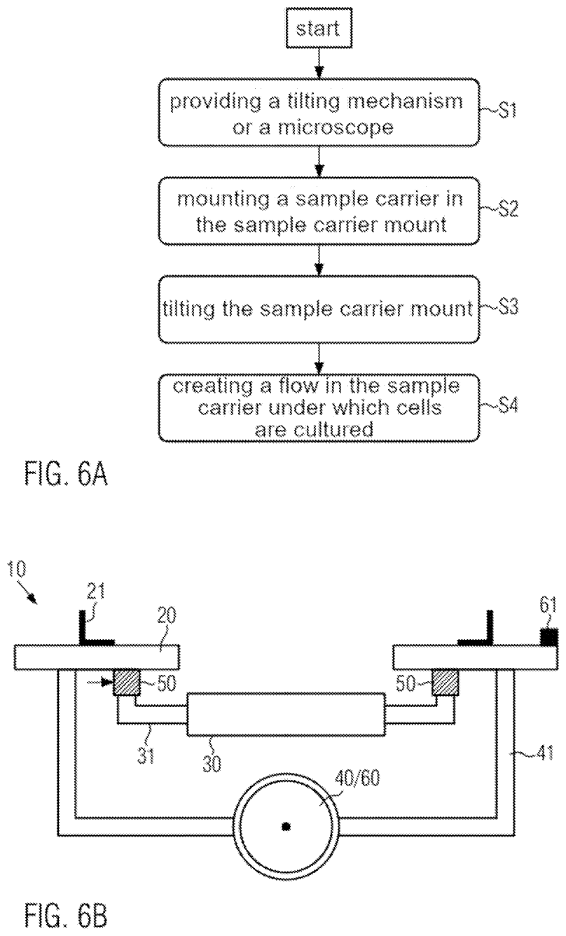

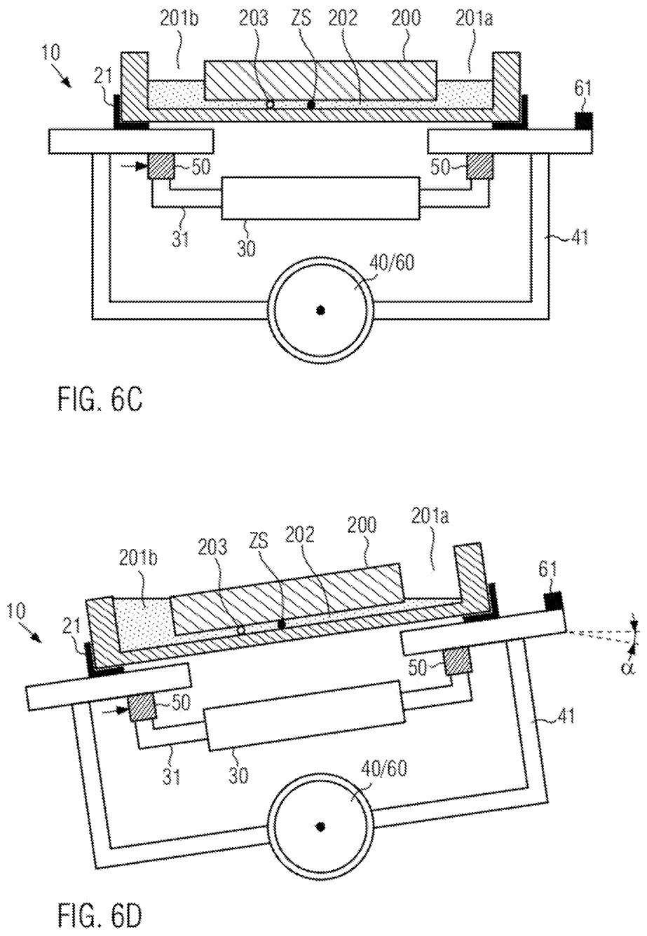

FIG. 6A show a flow chart for a method for microscopy; and

FIGS. 6B to 6D show a method for microscopy.

Hereinafter and in the figures, the same reference characters shall be used for the same or corresponding elements in the various embodiments, unless otherwise specified.

FIG. 1A shows a cross section of a tilting mechanism 10 according to the invention for microscopy according to a first embodiment. Tilting mechanism 10 there comprises a sample carrier mount 20 and an optical component 30. Optical component 30 is shown only schematically in this example. Optical component 30 can be, for example, an objective, where the objective can be a collector lens or a lens system comprising a plurality of lenses. The optical component is connected to sample carrier mount 20 by way of a holder 31. In this example, this connection is in particular rigid so that no relative motion between optical component 30 and sample carrier mount 20 is possible.

Sample carrier mount 20 additionally comprises an insertion rail 21 into which a sample carrier can be inserted and optionally affixed. Insertion rail 21 can be formed circumferentially on the upper side of sample carrier mount 20. It is also possible for insertion rail 21 to be formed only at specific sections on the upper side of the sample carrier mount, for example, such that the corners of the sample carrier abut against insertion rail 21. In this example, the base side of sample carrier mount 20 can be identified as its upper side at which insertion rail 21 is also attached and on which a sample carrier can be inserted.

Sample carrier mount 20 is connected by way of a holding device 41 to a joint 40 and at the same time is mounted to be tiltable about this joint 40. Joint 40 can be, for example, a ball joint or a pivot joint (hinge joint). In the case of a pivot joint, the tilt axis runs in the plane of the drawing and through the center of joint 40. With a ball joint, the tilt axis is accordingly disposed in a horizontal plane that runs through the center of joint 40.

FIG. 1A finally shows optical axis OA of optical component 30 Optical axis OA is oriented perpendicular to the base side of sample carrier mount 20 described above and runs through the geometric center and in particular through the center of curvature of optical component 30. In the example shown, optical axis OA is additionally oriented perpendicular to the previously described tilt axis, the orientation is always maintained in tilting mechanism 10.

The tilting mechanism described from FIG. 1A is shown in the tilted state in FIG. 1B, where the tilt is executed about joint 40 and about a tilt axis which runs into the plane of the drawing and through the center of joint 40. Since optical component 30 and sample carrier mount 20 are connected rigidly to one another, they were tilted in the same way so that optical axis OA of the optical component is aligned perpendicular to the base side of sample carrier mount 20, in particular even after tilting.

Tilting mechanism 10 was tilted by angle α with respect to the horizontal alignment of the tilting mechanism, as in FIG. 1A, and about the tilt axis described. The horizontal alignment can be regarded to be the initial position of tilting mechanism 10.

A further embodiment of a tilting mechanism 10 is shown in FIG. 2A. This figure shows a cross section of tilting mechanism 10 in a front view. As is already known from the embodiment according to FIG. 1A, tilting mechanism 10 comprises a sample carrier mount 20 with an insertion rail 21 and an optical component 30. In this case, optical component 30 consists of an objective and a mirror, where the objective is drawn only schematically for the reasons mentioned above. The mirror is mounted in a mirror mount 32. In a simple form, mirror mount 32 is a plate with a recess, where the mirror is mounted in this recess. However, mirror mount 32 is not restricted to this example.

Mirror mount 32 is rotated relative to the base side of sample carrier mount 20, where the axis of rotation of the mirror and mirror mount 32 is parallel to the base side of sample carrier mount 20 and lies in the plane of the drawing. In particular, the angle of rotation is 45 degrees so that light passing through the objective along the optical axis is deflected at a right angle and emerges perpendicular from the plane of the drawing.

Tilting mechanism 10 is configured overall such that a tilt axis 33 is arranged at mirror mount 32 (not shown in this figure but in FIG. 2B) and runs centrally through the mirror. Tilting mechanism 10 is therefore mounted to be rotatable about this tilt axis 33. Tilt axis 33 points perpendicularly out of the plane of the drawing and is also perpendicular to optical axis OA. Tilting mechanism 10 can be tilted clockwise or counterclockwise as shown by the arrows.

It is possible with this embodiment of tilting mechanism 10 to perform a tilt without the optical path of the light downstream of optical component 30 changing. This is for the reason that the center of rotation of tilting mechanism 10 is disposed at the point of the mirror, whereby the point of incidence and angle of incidence of light on the mirror remain constant regardless of a tilt of tilting mechanism 10.

A tilting mechanism 10 according to a further embodiment is shown in cross section and in a side view in FIG. 2B. There, a mirror alone forms optical component 30. The mirror is mounted in a mirror mount 32 and the surface of the mirror is rotated relative to the base side of sample carrier mount 20, in particular by 45 degrees. Mirror mount 32 further comprises a tilt axis 33 which, as described with reference to FIG. 2A, is aligned perpendicular to the optical axis and centrally through the mirror. In addition, tilt axis 33 lies in the plane of the drawing in this side view. In particular, the arrangement of tilt axis 33 is identical in the two examples according to FIGS. 2A and 2B. A tilt of tilting mechanism 10 is produced by a rotation about tilt axis 33, for example, about a corresponding joint 35, where the direction of rotation is represented by the arrows.

Objective 34 is not part of tilting mechanism 10 and is in particular fixedly mounted so that it does not move independently of a tilt of tilting mechanism 10. This arrangement has the advantage that the objective, which is of great importance for generating an image, does not have to be moved. This suppresses aberrations that are induced by a relative motion between the mirror and the objective. Objective 34 can be in particular part of a microscope, in particular if tilting mechanism 10 is integrated into an existing microscope.

FIG. 3A shows a tilting mechanism 10 comprising a motion device 50 as a top view, where motion device 50 is configured such that it moves optical component 30 in relation to the affixed sample carrier mount 20. In the specific case, motion device 50 is a translation stage in which optical component 30 can be moved in a plane parallel to the base side of the sample carrier mount. The translation stage can be a conventional manual model comprising a set screw 51, a return spring 52, and a readable scale 53 for each of the two axes. Optical component 30 can be moved along the associated axis by rotating set screw 51. Since there are two axes arranged perpendicular to one another, each with a set screw 51, which form a two-dimensional translation stage, optical component 30 can be moved horizontally in a plane and relative to sample carrier mount 20.

Due to a comparable shape of the motion device, sample carrier mount 20 can likewise be mounted to be movable and moved by motion device 50 while optical component 30 is fixed.

As in the embodiments described above, the tilting mechanism furthermore comprises an insertion rail 21 on the upper side of sample carrier mount 20, where insertion rail 21 is configured for a rectangular sample carrier and affixes the sample carrier at the four corners, and a holder 31 for optical component 30.

FIG. 3B shows a further embodiment for motion device 50, with which it is possible to additionally move optical component 30 parallel to its optical axis OA. Components analogous to the embodiment in FIG. 3A shall presently not be explained again, but only the differences shall be highlighted.

The section of tilting mechanism 10 shown comprises a base plate 55 which is arranged in a fixed manner in relation to sample carrier mount 20. Arranged symmetrically between base plate 55 and holder 31 of the optical component are motors 54 or comparable drive devices which perform a vertical motion along optical axis OA of optical component 30. In order for optical component 30 to be movable along optical axis OA, two motors 54 must be driven synchronously. This can be, for example, a piezoelectric actuator or an electric motor. In particular, the motion device presently shown can be combined with that embodiment from FIG. 3A so that the motion device enables a relative motion between sample carrier mount 20 and optical component 30 along all three directions in space.

A tilting mechanism 10 according to a further embodiment is shown in FIG. 4. It first comprises some components of the first embodiment according to FIG. 1A so that only the differences shall be explained hereafter.

Tilting mechanism 10 shown has, firstly, a motion device 50 which is configured such that it can move optical component 30 in the vertical direction or along optical axis OA, respectively. It is to be noted that optical component 30 by design must perform tilting in the same manner, specifically about the same axis (or a coaxial axis) as sample carrier mount 20, but the two components are not rigidly connected to one another because a relative motion can take place between the components. The motion device comprises a holder 31 for optical component 30 and a motor, for example, an electric motor or a piezoelectric actuator, which can perform the motion of motion device 50.

Motion device 50 is actuated by an electronic control device 58 which regulates the motion of the motor. Electronic control device 58 comprises a signal input 55, a processor 56, and a signal output 57. For example, a target coordinate for optical component 30 can be entered into the signal input. The input can be done in associated control software or on an integrated keyboard or a similar device. This input is then used by processor 56 to generate a control signal therefrom. This control signal is output by electronic control device 58 through signal output 57 and is transmitted to motion device 50. Motion device 50 then moves optical component 30 by way of the motor so that it assumes the target coordinates specified.

A tilt angle of tilting mechanism 10 can be controlled by way of an inclination device 60. Inclination device 60 can be, for example, a rotary motor, in particular an electrically controllable one. The rotary motor there also comprises joint 40 about which the tilting is executed.

Furthermore, tilting mechanism 10 has an inclination sensor 61 which detects the tilt angle of tilting mechanism 10 in relation to a horizontal initial position. For example, capacitive or magnetoresistive fluid inclination sensors are suitable. Inclination sensor 61 outputs a specific value for the tilt angle or another measured value that depends on the tilt angle and transmits it to control device 70.

Similar to electrical control 58, control device 70 comprises a signal input 71, a processor 72, and a signal output 73. The value that is output by inclination sensor 61 is entered into signal input 71. A specific target value for the tilt angle to which tilting mechanism 10 is to be set can also be entered by way of signal input 71. Processor 72 uses this value and the specified target value and from this generates a control signal which is output from signal output 73 and transmitted to Inclination device 60. In the example shown, the control signal is transmitted directly to the rotary motor as a component of Inclination device 60. The rotary motor receives the control signal and produces a tilt of tilting mechanism 10 so that the tilt angle is adjusted to the predetermined target value.

The control process of the tilt angle can be configured in particular as a feedback loop. For this purpose, the tilt angle is controlled continuously in that inclination sensor 61 continuously outputs a measured value and control device 70 correspondingly continuously generates a control signal and transmits it to Inclination device 60 in order to maintain tilting mechanism 10 at a specific tilt angle. This allows the tilt angle to be kept particularly stable over long periods of time.

Electronic control 58 and control device 70 can be comprised by a control device 80 which can carry out the entire control process described of the tilting mechanism. Control device 80 can there have several signal inputs, processors, and signal outputs which are connected to the respective devices of tilting mechanism 10.

FIG. 5A illustrates in cross section the schematic structure of a microscope 100 according to the invention, in the case shown an inverted transmitted light microscope. Microscope 100 there comprises a tilting mechanism 10 described and a projection system 105. A stand 101 customary for such a microscope serves as a holder and stabilizing element.

Microscope 100 can in principle be equipped with the tilting mechanisms explained in this specification so that it is illustrated reduced only to the relevant components in this figure, namely sample carrier mount 20 with an insertion rail 21, optical component 30, and an inclination device 60. Optical component 30 can be in particular an objective, but is presently only shown schematically.

Microscope 100 can there have a modular structure, in particular tilting mechanism 10 can be a replacement member that can be subsequently integrated into microscope 100. In this case, different embodiments of tilting mechanism 10 can be compatible with microscope 100.

Projection system 105 comprises several mirrors 110 and a projection lens 120 which in the present example is configured in the form of an eyepiece. The projection lens is mounted in a rail 121 so that projection lens 120 is mounted to be movable along its optical axis. As with optical component 30 of tilting mechanism 10, the optical axis of projection lens 120 also passes through the center of curvature and is orthogonal to the remaining axes of symmetry. Projection system 105 furthermore comprises an imaging component 130. The imaging component can be, for example, a camera with an LCD chip or another imaging sensor that can be mounted at microscope 100 for electronically generating and outputting an image.

Mirrors 110 are used to direct the light from optical component 30 through projection lens 120 onto imaging component 130 and are therefore referred to as deflection mirrors. In the example shown, three mirrors 110 are used for this purpose, but a microscope 100 according to the invention is not restricted to this number and configuration. In addition, further optical elements for beam guidance can be present along the optical path, for example, a prism.

Likewise shown in FIG. 5A is the optical path of light propagating through microscope 100. Starting from a light source above the microscope (e.g. a condenser, not shown), the light first passes through a sample carrier that can possibly be mounted in sample carrier mount 20 and thereafter through optical component 30. As a result of a tilt of tilting mechanism 10, the refraction of light in the optical component 30 changes and consequently also the optical path through microscope 100. Therefore, the point of incidence and the angle of incidence on projection lens 120 and imaging component 130 change. This change can be compensated for by mirrors 110 of projection system 105. For this purpose, mirrors 110 are mounted to be movable and can be rotated such that said point of incidence and angle of incidence remain unchanged. The adjustment of mirrors 110 can be performed manually or, as explained hereafter, by a control device provided for this purpose.

If the length of the optical path changes greatly, an image formed by projection lens 120 may become out of focus. In this case, projection lens 120 is mounted to be movable in a rail 121 along its optical axis so that the distance between the projection lens and imaging component 130 can be adjusted and a sharp projection can be obtained. Alternatively, the lens can be set to infinity such that a change in the length of the optical path between the objective and projection lens 120 does not result in a shift in the image plane.

FIG. 5B shows a detailed view of microscope 100 from FIG. 5A in which projection system 105 is explained in more detail. Two of mirrors 110 contained each comprise a motor 111 with which mirrors 110 can be rotated in order to compensate for a described change in the point of incidence and the angle of incidence on projection lens 120 and imaging component 130. These can be mirrors that are mounted in a conventional piezo-driven mirror mount (motorized mirror mount), as is common in the context of such optical applications.

Furthermore, projection lens 120 is mounted in a motorized rail 121 with which projection lens 120 can be moved electronically along its optical axis. Motors 111 and rail 121 are controlled by an electronic control device 140 comprising a signal input 141, a processor 142, and a signal output 143. The signal input can be provided, for example, by imaging component 130 which detects that a point of incidence and/or an angle of incidence on the component is/are no longer correct and/or that the image is no longer in focus. The signal can then be, for example, an error signal that reflects the deviation in the angle of incidence and/or the point of incidence. This signal is received by signal input 141 and transmitted to processor 142 which uses it to calculate a necessary correction of the mirror positions and/or the position of projection lens 120. This correction signal is output from signal output 143 and transmitted to motors 111 and/or rail 121 where the respective correction of the optical path and/or the focusing of projection lens 120 is performed. Control device 140 can be in particular equipped with a feedback mechanism so that the correction described can occur continuously. In addition, control device 140 can be part of external control device 80, i.e. can be integrated therein.

FIG. 5C shows a microscope 100 of the invention according to a further embodiment. It differs from the embodiment of FIG. 5A in the configuration of tilting mechanism 10, optical component 30, and projection system 105. These differences shall be discussed in more detail hereafter.

First, a tilting mechanism 10 as shown in FIG. 2B is used. Tilting mechanism 10 there comprises a mirror as optical component 30. The mirror is connected, in particular connected rigidly, to sample carrier mount 20 by way of a corresponding mechanism. Tilt axis 33 of the mirror and therefore also of entire tilting mechanism 10 is aligned such that it runs parallel to the base side of sample carrier mount 20 and through the geometric center of the mirror. Tilting mechanism 10 is then tilted about said tilt axis 33.

As already described above, the embodiment of tilting mechanism 10 allows for the optical path of the light not to change after reflection from the mirror despite tilting. It is therefore possible to combine the tilting mechanism with an objective 34 integrated into microscope 100. In particular, it does not have to be readjusted in the event of tilting, which enables particularly efficient microscopy. Objective 34 can there in principle be mounted to be movable in order, for example, to adjust its position along the optical path so that a sharp image is generated at the beginning of a microscopic examination.

The projection system comprises two deflection mirrors 110, a projection lens 120 mounted to be movable in a rail, and an imaging component 130. The same considerations apply as in the preceding embodiment of FIG. 5A in particular with regard to projection lens 120 and imaging component 130.

A microscope 100 of the invention according to a further embodiment shall be explained with reference to FIG. 5D. Therein, microscope 100 also comprises a tilting mechanism 10 described which does not need to be explained in more detail at this point, and a stand 101. Furthermore, microscope 100 comprises a projection system 105 with a projection lens 120 and an imaging component 130, in particular a camera with a CCD chip or a similar device.

Sample carrier mount 20 is connected to stand 101 by way of a holder 102 and is mounted to be rotatable or tiltable in relation to stand 101. The rotation or tilt, respectively, takes place about an axis which is aligned coaxially relative to holder 102.

Optical component 30, projection lens 120, and imaging component 130 are connected to one another by a tube 125 that is closed in particular around the circumference. The connection there is such that the three components mentioned cannot move relative to one another. Tube 125 not only fulfills the function of a stable holder, but also prevents light, for example scattered light, from entering imaging component 130 from the side. This ensures a high projection quality.

Tube 125 is connected to sample carrier mount 20 so that, when the entire system composed of tilting mechanism 10 and projection system 105 performs a common tilt motion, the optical path through the system does not change as a result of a tilt. Corrections to the optical path, for example, using deflection mirrors, are therefore not necessary.

Alternatively, objective 30 can be mounted to be movable along the vertical axis to change the distance between objective 30 and sample carrier mount 20. In this case, objective 30 can be in particular set to infinity so that projection lens 120 does not have to be adjusted to a changed position of objective 30.

The explanations given above apply with regard to the implementation of a tilt. For example, the tilt can take place in a motorized manner, where, for example, holder 102 can be rotated by a suitably configured motor. The same considerations also apply to a possible movability of the objective. This as well can be implemented by a suitably configured motor. It is furthermore possible for the tilting mechanism comprised in microscope 100 to comprise a motion device with the aforementioned properties.

Imaging component 130 is connected in particular in an electronic manner to a display device 150, for example, a monitor, which can be part of microscope 100, for transmitting an electronic signal from imaging component 130 to display device 150. The images generated by imaging component 130 are displayed to a user by display device 150.

FIG. 6A shows a method according to the invention for microscopy as a flow chart. In first step S1 of the method, a tilting mechanism 10 presently described or a microscope 100 presently described is provided. In principle, the method can be carried out with all of the embodiments presented here.

In subsequent step S2, a sample carrier 200 is mounted in sample carrier mount 20 of tilting mechanism 10. Sample carrier 200 there comprises a first reservoir, a second reservoir, and a channel which connects the first reservoir to the second reservoir. A fluid and cells or a cell structure are disposed in sample carrier 200, where cells can be distributed in a reservoir, a channel, or at several locations in the sample carrier.

Now, in step S3, sample carrier mount 20 is tilted, where no restrictions are basically provided for the tilt with regard to the tilt angle and/or the tilt axis. In particular, tilts are performed at an angle between 0 degrees and 20 degrees. This tilt creates a flow by gravity due to a fluid transport from the first reservoir through the channel into the second reservoir.

The cells in sample carrier 200 are cultured under this flow (step S4).

The method of microscopy described above shall be illustrated in more detail with reference to FIGS. 6B to 6D.

In the first step of the method, as shown in FIG. 6B, a tilting mechanism 10 as described above is provided. Tilting mechanism 10 is shown in cross section as a front view. However, the method can also be carried out in the same way with a microscope 100 described comprising tilting mechanism 10. According to the invention, tilting mechanism 10 comprises a sample carrier mount 20 and an optical component 30. In addition, sample carrier mount 20 has an insertion rail 21, and optical component 30 has a holder 31 which rigidly connects sample carrier mount 20 and optical component 30 to one another. According to the embodiment described above, tilting mechanism 10 furthermore comprises a motion device 50 and an inclination device 60 which at the same time serves as a joint 40 about which tilting takes place. Reference is made to the associated embodiment in FIG. 4 for a more detailed explanation of the components.

In the subsequent method step (see FIG. 6C), a sample carrier 200 is now introduced into sample carrier mount 20 and mounted therein. For this purpose, sample carrier 200 is inserted into insertion rail 21 which can be adapted precisely to the size of the sample carrier. In order to furthermore prevent the sample carrier from being able to move within insertion rail 21 or in sample carrier mount 20 in general, further fastening devices can be present. For example, a locking screw is available for this purpose which passes laterally through insertion rail 21 and presses sample carrier 200 against insertion rail 21 and thereby affixes it. This can be advantageous because sample carrier 200 can easily slip in a sample carrier 20 that is not ideally adjusted as a result of tilting, and the microscopic examination is significantly impaired as a result.

The sample carrier itself comprises a first reservoir 201a, a second reservoir 201b, and a channel 202 which connects first reservoir 201a to second reservoir 201b. The terms first reservoir 201a and second reservoir 201b can be used interchangeably since they are not structurally different from each other. A fluid, in particular a cell medium, as well as cells or cell structures ZS are disposed in sample carrier 200. They can be introduced into sample carrier 200 as part of the method or can already be present in sample carrier 200. The cells themselves can be disposed at a specific location within channel 202 as shown, but they can also be arranged in a reservoir 201a, 201b or be distributed over a larger region within sample carrier 200.

In addition, the sample carrier comprises a fluid sensor 203 which in this example is arranged within channel 202 and is configured in such a way as to detect a flow speed within the channel. The value determined can then be output electronically.

As shown in FIG. 6D, tilting mechanism 10 and therefore also sample carrier mount 20 and optical component 30 are subsequently tilted by an angle α. To execute the tilt, an inclination device 60 is used like in the embodiment of a tilting mechanism 10 described above.

As a result of the tilt, the fluid is transported from first reservoir 201a in the direction of second reservoir 201b. This transport of fluid creates a flow, in particular at the location of the cells, where the cells are cultured under this flow. Tilting mechanism 10, as described, allows for precise adjustment and control of the tilt angle and thereby of the flow. In particular, fluid sensor 203 which transmits an associated measured value to control device 70 can be used for this purpose. The processor can determine a target value for the tilt angle from the flow rate measured and, from this, a control signal that is respectively transmitted to inclination device 60. The inclination device thereby adjusts the tilt angle accordingly. This method can also be carried out in the same way with a fluid level measured or a flow direction measured, where fluid sensor 203 must be configured accordingly.

The method described can be extended beyond the steps described by varying the tilt angle over time. For this purpose, inclination device 60 must be configured such that it can adjust tilt angle α continuously. For example, the tilt angle can be changed linearly or periodically, such as sinusoidally, over time. In particular, the sign of the tilt angle is there changed as a change in the tilt direction so that the direction of flow within sample carrier 200 is reversed.

A continuous flow of the cell medium in sample carrier 200 can be generated by this method described without there being a need for external devices because the fluid can be kept moving continuously by periodically reversing the tilt direction. Furthermore, the tilt angle can be continuously controlled and adjusted, for example, using a feedback mechanism of inclination device 60 in interaction with a fluid sensor and an inclination sensor 61.

The method described is not restricted to the combination of a tilting mechanism 10 and a sample carrier 200 shown. In particular, the method can be carried out with all tilting mechanisms 10 and microscopes 100 presented in this description. Likewise, the combination of features of sample carrier 200 is not restrictive. Sample carriers are suitable for the method provided they have two reservoirs 201a, 201b connected by a channel 202 and can be used with tilting mechanism 10 or microscope 100 in a meaningful manner. In addition to the components mentioned, sample carrier 200 can also have, for example, micro-filter structures or valves.

1. Tilting device (10) for microscopy, comprising:
a sample carrier mount (20), and
an optical component (30) with an optical axis (OA),
where said optical axis (OA) is aligned with respect to a base side of said sample carrier mount (20),
where said sample carrier mount (20) and said optical component (30) are mounted to be tiltable, and
where the alignment of said optical axis (OA) with respect to said base side of said sample carrier mount (20) is maintained when said sample carrier mount (20) and said optical component (30) are tilted.

2. Tilting device (10) according to claim 1, where said optical component (30) is an objective and/or a mirror.

3. Tilting device (10) according to claim 1, where said sample carrier mount (20) and said optical component (30) are each connected to a joint (40) and are mounted to be tiltable about said respective joint (40).

4. Tilting device (10) according to claim 1, where said tilting mechanism (10) is configured such that said sample carrier mount (20) and said optical component (30) are tilted about a tilt axis (33) which is aligned to be perpendicular to said optical axis (OA).

5. Tilting device (10) according to claim 1, where said sample carrier mount (20) and said optical component (30) can be tilted by a tilt angle between 0 degrees and 1 degree, in particular between 0 degrees and 20 degrees, with respect to a horizontal position of said sample carrier mount (20).

6. Tilting device (10) according to claim 1, furthermore comprising a motion device (50),
where said motion unit (50) is configured such that it can move said optical component (30) in a plane parallel to said base side of said sample carrier mount (20), or
where said motion unit (50) is configured such that it can move said optical component (30) perpendicular to said base side of said sample carrier mount (20), or parallel to said optical axis (OA), respectively.

7. Tilting mechanism according to claim 6, where said motion unit (50) is configured such that it can move said sample carrier mount (20) in a plane perpendicular to said optical axis (OA) of said optical component (30), or
where said motion device (50) is configured such that it can move said sample carrier mount (20) parallel to said optical axis (OA).

8. Tilting device (10) according to claim 1, furthermore comprising an inclination unit (60) with a motor (13),
where said motor (13) is connected to said optical component (30) and/or to said sample carrier mount (20), and
where said motor (13) is configured such that it tilts said sample carrier mount (20) and said optical component (30).

9. Tilting device (10) according to claim 8, furthermore comprising:
an inclination sensor (61), and
a control unit (70),
where said inclination sensor (61) is configured such that it can detect and output a value of a tilt angle of said tilting mechanism (10), and
where said control unit (70) is configured to receive the value output by said inclination sensor (61) and, based on the value received and a predetermined target value, to transmit a control signal to said inclination unit (60) so that said inclination unit (60) adjusts the tilt angle of said tilting mechanism (10) to said predetermined target value.

10. Microscope (100) comprising,
a tilting device (10) according to claim 1,
a stand (101), and
a projection system (105) with a projection lens (120), in particular an eyepiece.

11. Microscope (100) according to claim 10, where said projection system (105) furthermore comprises:
one or more mirrors (110), where said mirrors (110) are mechanically coupled or controllable electronically individually, and/or
an imaging component (130).

12. Microscope (100) according to claim 10 one of the claim 10 or 11,
where said projection lens (120) is mounted to be movable along its optical axis, and/or where a focal length of said projection lens (120) is variable, and
where a motion of said projection lens (120) along its optical axis and/or said focal length can be controlled individually.

13. Method for microscopy comprising:
providing a tilting device (10) for microscopy according to claim 1;
mounting a sample carrier (200) in said sample carrier mount (20); and
tilting said sample carrier mount (20),
where said sample carrier (200) comprises a first reservoir (201a), a second reservoir (201b), and a channel (202),
where said first reservoir (201a) is connected to said second reservoir (201b) by way of said channel (200),
where a fluid is transported from said first reservoir (201a) into said second reservoir (201b) by tilting said sample carrier mount (20),
where a flow is produced in particular by the transport of said fluid, and where cells (ZS) are cultured under this flow.

14. Method according to claim 13,
where a tilting device (10) according to claim 8 is provided, and
where said sample carrier (200) furthermore comprises a fluid sensor (203),
where said fluid sensor (203) detects and outputs a value of one or more of a fluid level, a flow rate, or a flow direction within said sample carrier (200) mounted in said sample carrier mount (20), and
where said control unit (70) receives the value that is output by said fluid sensor (203) and, based on the value received, transmits a control signal to said inclination unit (60), so that said inclination device (60) adjusts the tilt angle of said tilting device (10) so that one or more of the fluid level, the flow rate, or the flow direction within said sample carrier (200) are adjusted to a predefined target value.

15. Use of a tilting device (10) for microscopy according to claim 1 for transporting a fluid within a sample carrier (200),
where said sample carrier (200) is mounted in said sample carrier mount (20),
where said sample carrier (200) comprises a first reservoir (201a), a second reservoir (201b), and a channel (202),
where said first reservoir (201a) is connected to said second reservoir (201b) by way of said channel (200), and
where a fluid is transported from said first reservoir (201a) into said second reservoir (201b) by tilting said sample carrier mount (20),
where a flow is produced in particular by the transport of the fluid, and where cells (ZS) are cultured under this flow.
